(19) United States
(12) Patent Application Publication
(10) Pub. No.: US 2024/0113063 A1
(43) Pub. Date: Apr. 4, 2024

(54) RADIATOR LAYERS FOR ULTRASONIC TRANSDUCERS

(71) Applicant: TEXAS INSTRUMENTS INCORPORATED, Dallas, TX (US)

(72) Inventors: Udit RAWAT, West Lafayette, IN (US); Bichoy BAHR, Allen, TX (US); Swaminathan SANKARAN, Allen, TX (US); Baher S. HAROUN, Allen, TX (US)

(21) Appl. No.: 17/957,446

(22) Filed: Sep. 30, 2022

(51) Int. Cl. H01L 23/00; A61B 8/00

(52) U.S. Cl. CPC H01L 24/24; A61B 8/4494; H01L 24/05; H01L 24/73; H01L 2224/05005; H01L 2224/2401; H01L 2224/73251

(57) ABSTRACT

In examples, a semiconductor die comprises a semiconductor substrate having a surface, the surface having first and second surface portions, and a radiator layer on the surface. The radiator layer comprises a metal member having a first metal member portion above the first surface portion and a second metal member portion above the second surface portion, a first distance between the first metal member portion and the first surface portion, and a second distance between the second metal member portion and the second surface portion, the first distance less than the second distance. The radiator layer includes first and second electrodes. The radiator layer includes a piezoelectric layer extending along a length of the radiator layer and on each of the first and second electrodes, the piezoelectric layer between the first and second metal members and the semiconductor substrate.

### BACKGROUND

Acoustic waves are useful in a variety of applications, including industrial and medical applications. In many such applications, a transducer converts electrical signals to acoustic waves, and the acoustic waves are provided to a target medium (e.g., the human body to view an organ, a semiconductor package to determine structural integrity). The acoustic waves reflect off features in the target medium and return to the transducer, which converts the acoustic signals to electrical signals. The electrical signals are subsequently processed by appropriate circuitry, such as a processor or microcontroller, to create images of those features.

### SUMMARY

In examples, a semiconductor die comprises a semiconductor substrate having a surface, the surface having first and second surface portions, and a radiator layer on the surface. The radiator layer comprises a metal member having a first metal member portion above the first surface portion and a second metal member portion above the second surface portion, a first distance between the first metal member portion and the first surface portion, and a second distance between the second metal member portion and the second surface portion, the first distance less than the second distance. The radiator layer includes first and second electrodes. The radiator layer includes a piezoelectric layer extending along a length of the radiator layer and on each of the first and second electrodes, the piezoelectric layer between the first and second metal members and the semiconductor substrate.

### BRIEF DESCRIPTION OF THE DRAWINGS

FIG. 1 is a block diagram of an electronic device having a radiator layer, in accordance with various examples.

FIG. 2 is a cross-sectional schematic diagram of an electronic device having a radiator layer, in accordance with various examples.

FIG. 3 is a cross-sectional schematic diagram of an electronic device having a radiator layer, in accordance with various examples.

FIG. 4 is a cross-sectional schematic diagram of an electronic device having a radiator layer, in accordance with various examples.

FIG. 5A is a cross-sectional schematic diagram of a semiconductor die having a radiator layer, in accordance with various examples.

FIG. 5B is a cross-sectional schematic diagram of a semiconductor die having a radiator layer, in accordance with various examples.

FIG. 5C is a top-down view of electrically active structures in a radiator layer of a semiconductor die, in accordance with various examples.

FIG. 5D is a top-down view of a piezoelectric layer in a radiator layer of a semiconductor die, in accordance with various examples.

FIG. 5E is a top-down view of metal members in a radiator layer of a semiconductor die, in accordance with various examples.

FIG. 6A is a cross-sectional schematic diagram of a semiconductor die and an acoustic wave emission pattern of the semiconductor die, in accordance with various examples.

FIG. 6B is a graph showing radiated power from a radiator layer as a function of operating frequency, in accordance with various examples.

FIG. 7 is a cross-sectional schematic diagram of a semiconductor die contacting a mold compound and an acoustic wave emission pattern of the semiconductor die and the mold compound, in accordance with various examples.

FIG. 8 is a cross-sectional schematic diagram of a semiconductor die on a matching layer and having a radiator layer, in accordance with various examples.

FIG. 9 is a cross-sectional schematic diagram of a semiconductor die having roughened surfaces, in accordance with various examples.

FIG. 10 is a cross-sectional schematic diagram of a semiconductor die having a patterned metal layer, in accordance with various examples.

### DETAILED DESCRIPTION

Ultrasonic devices, such as medical ultrasound machines, include semiconductor dies coupled to ultrasonic transducers that excite acoustic waves. The ultrasonic transducer directs the acoustic waves toward a subject external to the ultrasonic device, such as human or animal tissue. Low-frequency (e.g., 100-300 MHz) acoustic wave technologies are particularly useful for extensive penetration of subjects being studied. For example, a low-frequency acoustic wave will penetrate human and animal tissue more deeply than will high-frequency acoustic waves. Semiconductor die radiators are generally unsuccessful in generating low-frequency acoustic waves, because the radiator architecture is not suited for controlling and directing such low-frequency acoustic waves. Thus, such radiators excite and emit acoustic waves in higher frequency ranges (e.g., 700 MHz), and these acoustic waves are unable to deeply penetrate the fluid, tissue, or other subject being studied. Further, the radiator architecture is not suited for providing a wide range of frequencies (e.g., a band of 100 MHz or more), and this lack of flexibility in acoustic wave frequency precludes higher resolution in imaging and limits the usefulness of the radiator in many applications.

This description describes various examples of an electronic device, such as an ultrasonic device, including a semiconductor die capable of exciting and emitting acoustic waves in lower frequency ranges (e.g., 100-300 MHz, inclusive) and over wider bandwidths (e.g., 100 MHz or more). More specifically, the semiconductor die includes a radiator layer structure having a piezoelectric layer in contact with multiple electrodes. The electrodes are alternatingly excited to generate an acoustic wave in the piezoelectric layer. As the radiator layer begins to resonate, metal members above the piezoelectric layer reflect frequencies in a target frequency range (e.g., 100-300 MHz, inclusive) and attenuate other frequencies outside of this range, thereby strengthening the acoustic waves in the target frequency range. The periodicity (e.g., lateral dimensions and/or pitch) of the electrodes and the vertical thicknesses of the various layers in the radiator layer determine the signal frequencies that are reflected and the signal frequencies that are attenuated. The electrodes and metal members are arranged in a pattern that precludes leakage of acoustic waves laterally, and instead, propagation of the acoustic waves is encouraged vertically through the semiconductor substrate. These acoustic waves exit the semiconductor substrate and enter the space external to both the semiconductor substrate and the electronic device containing the semiconductor die. Because the radiation layer is structurally configured to produce and emit low-frequency acoustic waves (e.g., 100-300 MHz, inclusive) and to do so over a wide range of frequencies (e.g., 100 MHz or more), the acoustic waves are able to penetrate deeply into the subject of study, deliver higher resolution images than would otherwise be available with lower bandwidths, and extend the application of acoustic wave technology to other applications (e.g., applications in which deep acoustic wave penetration would be useful) than would be possible at higher frequencies and/or lower bandwidths.

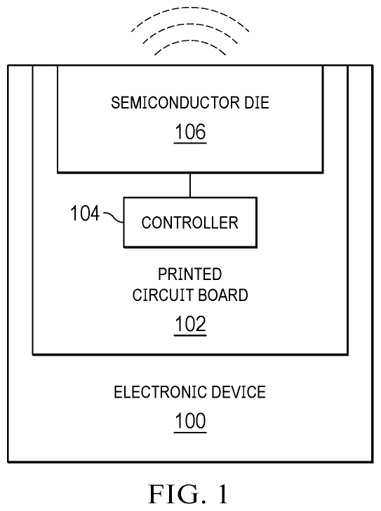

FIG. 1 is a block diagram of an electronic device 100 having a radiator layer, in accordance with various examples. The electronic device 100 may be any device in which acoustic wave technology is useful. Example electronic devices 100 include medical ultrasound devices, navigation devices, communication devices, imaging devices, cleaning devices and mixing devices. The description below assumes the electronic device 100 has a medical ultrasound application, but the scope of this description is not limited to medical ultrasound applications. The electronic device 100 includes a printed circuit board (PCB) 102. The electronic device 100 also includes a controller 104 and a semiconductor die 106. The controller 104 and the semiconductor die 106 may be coupled to the PCB 102. In examples, the semiconductor die 106 is arranged to be flush with, or within 5 millimeters of, a surface of the electronic device 100 to facilitate the emission and reception of acoustic waves into and from a subject being studied (e.g., human or animal tissue). As described in detail below, the semiconductor die 106 includes a semiconductor substrate and a radiator layer on the semiconductor substrate. The radiator layer is configured to excite and emit acoustic waves through the semiconductor substrate. Further, as described below, the structure of the radiator layer is adapted to reflect acoustic waves in relatively lower frequency ranges (e.g., 100 MHz to 300 MHz, inclusive) and across a larger bandwidth (e.g., at least 100 MHz), thereby mitigating the challenges associated with other solutions, as described above.

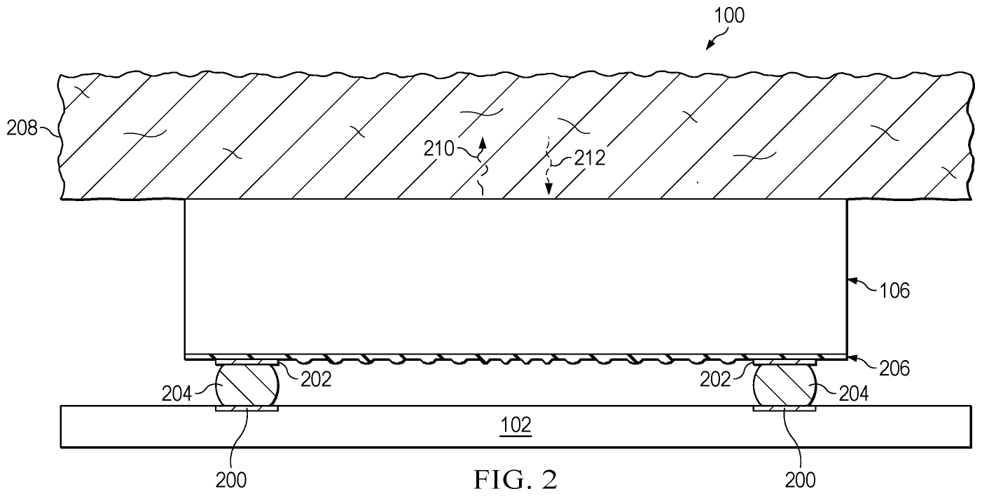

FIG. 2 is a cross-sectional schematic diagram of the electronic device 100 having a radiator layer, in accordance with various examples. The example electronic device 100 of FIG. 2 includes the PCB 102 and the semiconductor die 106 coupled to the PCB 102. The electronic device 100 also may include the controller 104 (FIG. 1), but the controller 104 is not expressly shown in FIG. 2. The PCB 102 includes metal bond pads 200 that are coupled to metal traces, which, in turn, are coupled to other components on the PCB 102, such as the controller 104. Further, the semiconductor die 106 includes a device side having metal bond pads 202 that are coupled to the bond pads 200 by conductive members 204, such as solder balls. The bond pads 202 are coupled to vias and other metallization (not expressly shown) in the semiconductor die 106, and these vias and metallization may be coupled to a radiator layer 206 on the device side of the semiconductor die 106. In this way, electrical pathways are formed between the radiator layer 206 and the controller 104 (FIG. 1), which enable the controller 104 to control the radiator layer 206 as described herein.

The portion of the semiconductor die 106 above the radiator layer 206 is the semiconductor substrate through which the radiator layer 206 emits and receives acoustic waves. Accordingly, the semiconductor substrate abuts a study subject 208 (e.g., human or animal tissue, fluids). The semiconductor die 106 emits acoustic waves 210 into the study subject 208 and receives reflected acoustic waves 212 from the study subject 208. In examples, acoustic waves are reflected responsive to a sharp impedance gradient between adjacent structures, such as between blood and bone. The semiconductor die 106, or another structure on the PCB 102 such as the controller 104, is configured to characterize the study subject 208 based on the acoustic waves emitted and received by the semiconductor die 106.

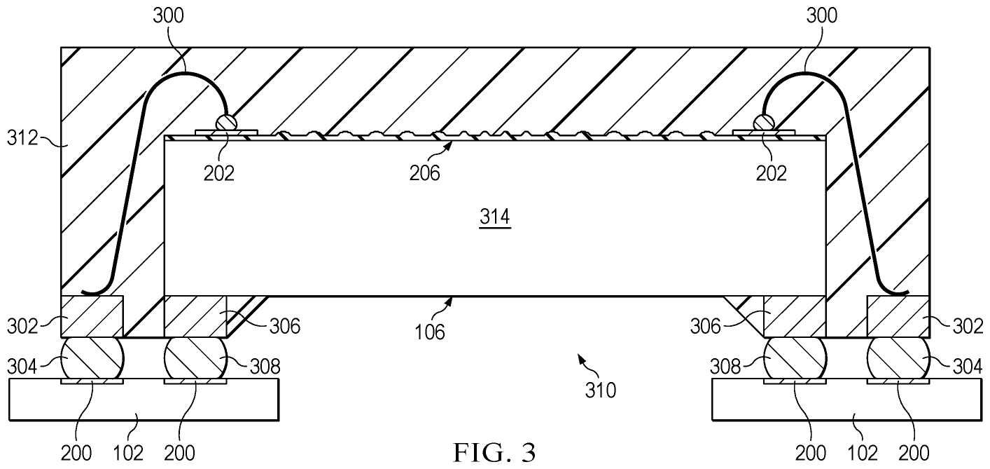

FIG. 3 is a cross-sectional schematic diagram of the electronic device 100 having a radiator layer, in accordance with various examples. The example electronic device 100 of FIG. 3 includes the PCB 102 and the semiconductor die 106. Other components, such as the controller 104 (FIG. 1), may also be coupled to the PCB 102 but are not expressly shown in FIG. 3. In contrast to the device side of the semiconductor die 106 of FIG. 2, the device side of the semiconductor die 106 of FIG. 3 faces away from the PCB 102. Thus, the bond pads 202 and the radiator layer 206 face away from the PCB 102. Accordingly, bond wires 300 couple the bond pads 202 to conductive members 302, and the conductive members 302 (e.g., leads of a lead frame) are coupled to the bond pads 200 of the PCB 102 by way of conductive members 304 (e.g., solder balls). The bond wires 300 are coupled to the conductive members 302 with any suitable type of bond, such as stitch bonds. Support members 306 are coupled to support members 308 (e.g., solder balls), which, in turn, are coupled to the PCB 102. In some examples, the support members 306 and 308 are non-conductive members. In some examples, the support members 306 and 308 are conductive members, with the support members 306 coupled to metallization within the semiconductor die 106 and the support members 308 coupled to bond pads 200 (not expressly shown under the support members 308) of the PCB 102. For example, the support members 306 and 308 may constitute a die attach pad and may operate as a ground plane. In some examples, the support members 306 and 308 are metallic and thus conductive, but are not coupled to bond pads or other metallization and are not configured to carry electrical signals. The PCB 102 includes an orifice 310 through which acoustic waves emitted by the semiconductor die 106 or reflected toward the semiconductor die 106 may pass. A mold compound 312 covers portions of the semiconductor die 106, the bond wires 300, the conductive members 302, and the support members 306. In examples, acoustic waves may extend through a semiconductor substrate 314, the mold compound 312, or both.

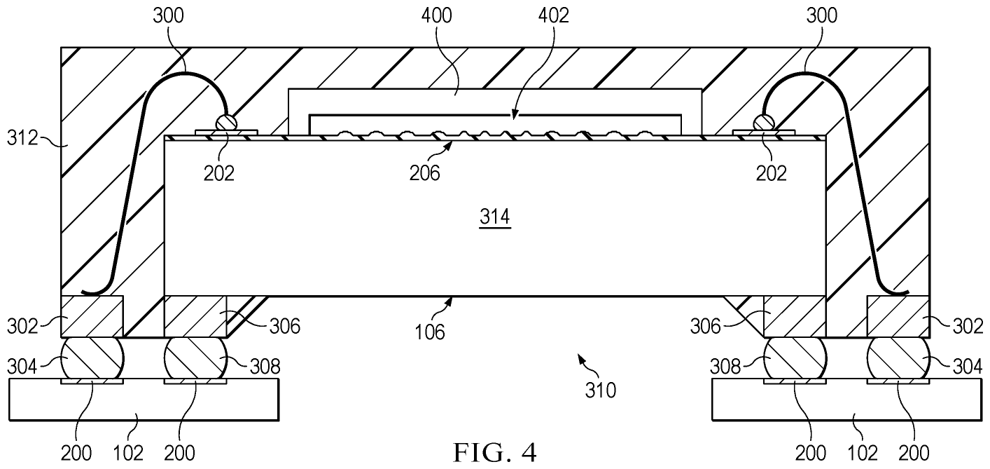

In some instances, blocking acoustic waves from passing through the mold compound 312 may be useful. FIG. 4 is a cross-sectional schematic diagram of the electronic device 100, in accordance with various examples. The electronic device 100 of FIG. 4 is identical to the electronic device 100 of FIG. 3, except that the electronic device 100 of FIG. 4 includes a cap 400 (e.g., composed of a semiconductor material such as silicon; a dielectric material; a metal or alloy) on the radiator layer 206. A cavity 402 formed of empty space (e.g., air) is between the semiconductor cap 400 and the radiator layer 206. The semiconductor cap 400 and the cavity 402 provide a sharp impedance gradient that blocks acoustic waves, thereby confining acoustic wave emissions and receptions to the semiconductor substrate 314.

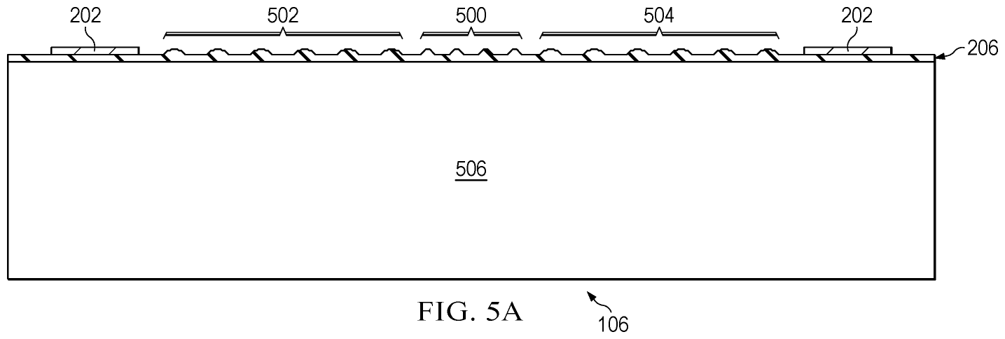

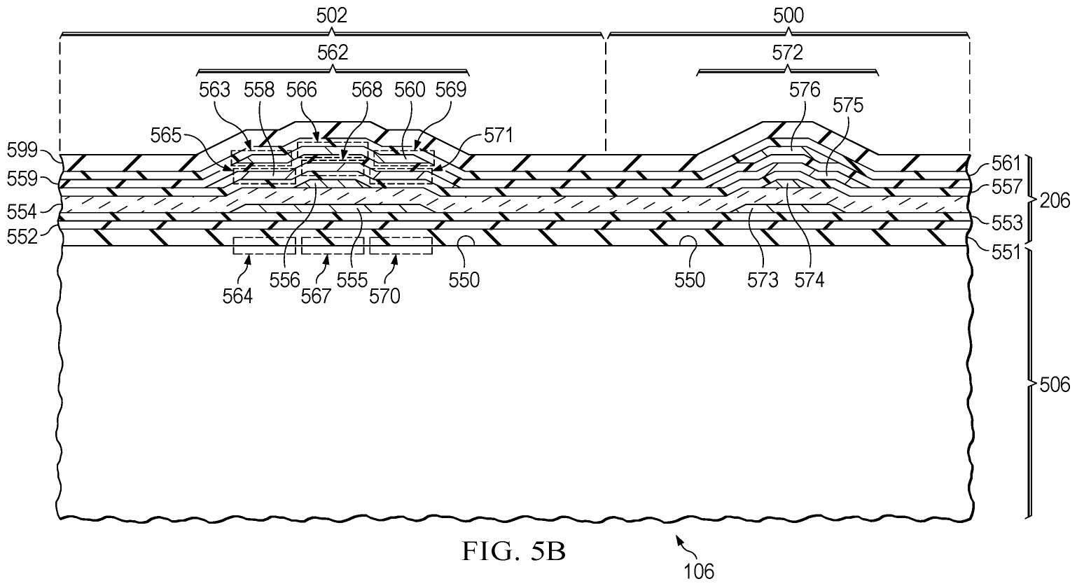

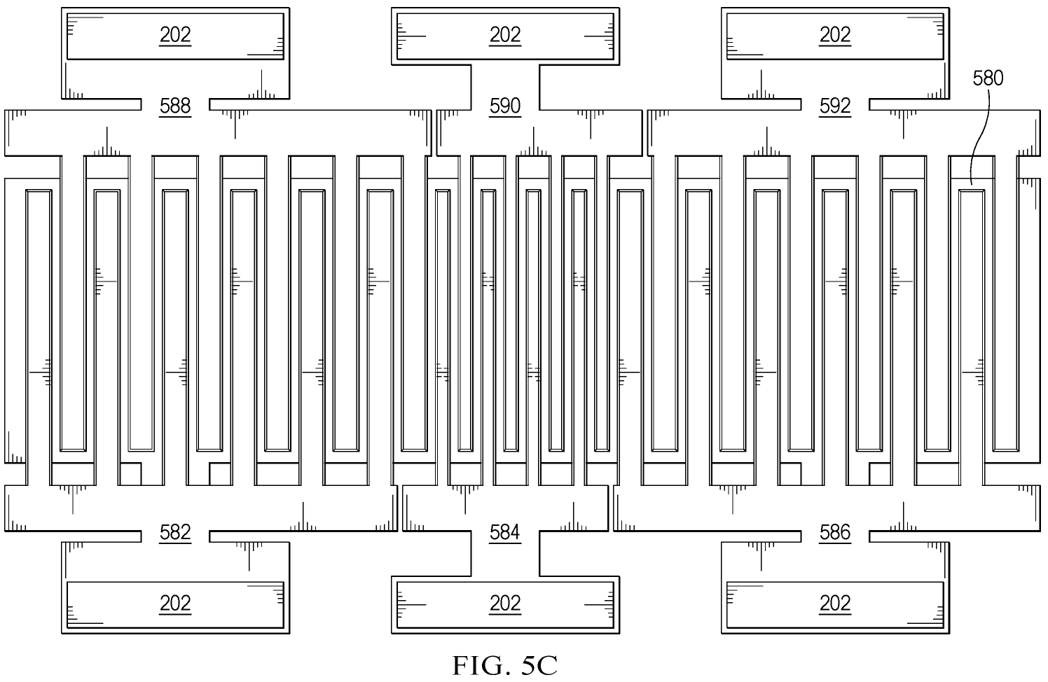

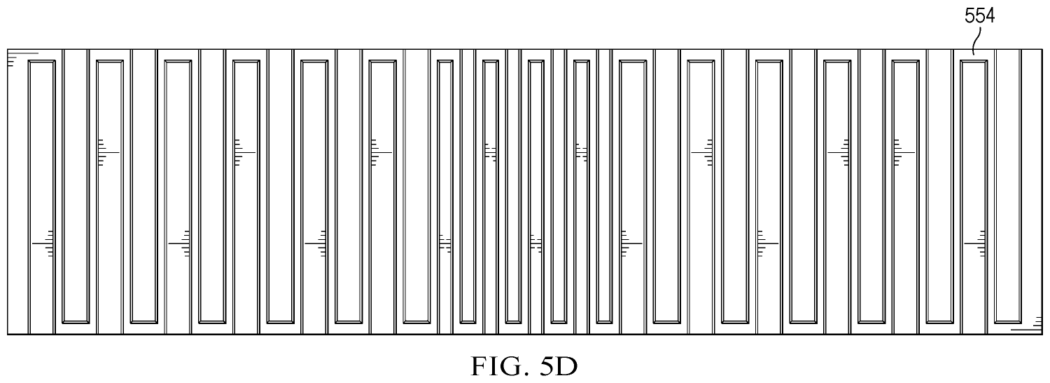

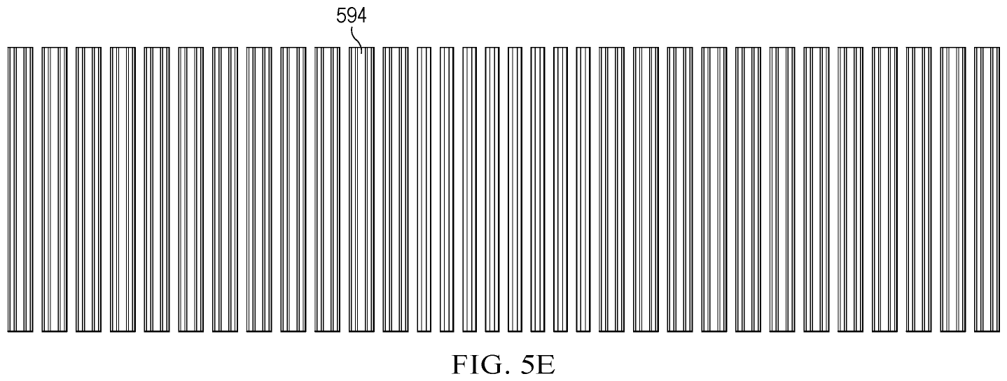

FIG. 5A is a cross-sectional schematic diagram of the semiconductor die 106 having a radiator layer, in accordance with various examples. The semiconductor die 106 includes the radiator layer 206. The radiator layer 206 includes a set of wave control structures 500 having a first pitch, a set of wave control structures 502 having a second pitch that is greater than the first pitch, and a set of wave control structures 504 having a third pitch that is greater than the first pitch. In some examples, the second and third pitches are approximately equal (within 10% of each other). In examples, the second pitch is 30%-40% greater than the first pitch, and in examples, the third pitch is 30%-40% greater than the first pitch. The semiconductor die 106 also includes the bond pads 202 between the set of wave control structures 502 and a closest edge of the semiconductor die 106, and also between the set of wave control structures 504 and a closest edge of the semiconductor die 106. The semiconductor die 106 also includes a semiconductor substrate 506 below the sets of wave control structures 500, 502, and 504. In examples, the semiconductor substrate 506 includes silicon, although other semiconductors, such as gallium nitride, are also possible.

The operation of the semiconductor die 106 is described in detail with reference to FIG. 5B, but generally, the controller 104 (FIG. 1) causes the radiator layer 206 to generate acoustic waves that resonate at an operating frequency range of 100 MHz to 300 MHz (e.g., 100 MHz to 200 MHz, inclusive, or 200 MHz to 300 MHz, inclusive), with a frequency band of 100 MHz or more. The sets of wave control structures 500, 502, and 504 are configured to reflect acoustic waves in the range of 100 MHz to 300 MHz, inclusive, and to attenuate acoustic waves of other frequencies. The greater pitch of the sets of wave control structures 502 and 504 (relative to the set of wave control structures 500) mitigates acoustic wave leakage in the lateral direction, and the lesser pitch of the sets of wave control structures 500 promotes acoustic wave propagation in the vertical direction (through the semiconductor substrate 506). This behavior is similar to that of radio-frequency waveguides and transmission lines, in which the periodicity (e.g., pitch) of the wave control structures (e.g., wave control structures 502, 504) in the radiator layer 206 enables the acoustic waves to propagate horizontally along the radiator layer 206. Changing the periodicity or pitch of the wave control structures changes the dispersion characteristics of the radiator layer 206. If electrodes in the wave control structures are excited in an alternating manner as described herein, the radiator layer 206 exhibits high-dispersion behavior and has a maximum acoustic wave frequency beyond which the radiator layer 206 will attenuate acoustic waves. Increasing the sizes (e.g., vertical thicknesses) of the wave control structures reduces this maximum acoustic wave frequency, and expanding the pitch between wave control structures in the radiator layer 206 will prohibit acoustic wave propagation.

FIG. 5B is a cross-sectional schematic diagram of the semiconductor die 106 having the radiator layer 206, in accordance with various examples. In particular, FIG. 5B shows neighboring wave control structures, with the left-hand wave control structure being from the set of wave control structures 502 (FIG. 5A), and the right-hand wave control structure being from the set of wave control structures 500 (FIG. 5A). The radiator layer 206 is on a surface 550 of the semiconductor substrate 506. The radiator layer 206 includes multiple structures in a stacked configuration. Specifically, the radiator layer 206 includes a dielectric layer 551 on the surface 550; a dielectric layer 552 on the dielectric layer 551; and a dielectric layer 553 on the dielectric layer 552. Other configurations, such as a single dielectric layer in lieu of the dielectric layers 551-553, also may be useful. A piezoelectric layer 554 is on the dielectric layer 553. The piezoelectric layer 554 extends along a length of the surface 550, and, more generally, along a length of the radiator layer 206. A pair of electrodes 555 and 556 is on opposing sides of the piezoelectric layer 554, as shown. The electrode 555 is positioned between the piezoelectric layer 554 and the dielectric layer 553, and the electrode 556 is positioned between the piezoelectric layer 554 and a dielectric layer 557. The dielectric layer 557 abuts the piezoelectric layer 554. A metal member 558 (e.g., composed of tungsten or tungsten-titanium) is on the dielectric layer 557, and a dielectric layer 559 is on the metal member 558. A metal member 560 (e.g., composed of tungsten or tungsten-titanium) is on the dielectric layer 559. A dielectric layer 561 is on the metal member 560, and a passivation layer 599 is on the dielectric layer 561.

As described, the piezoelectric layer 554 extends along a length of the radiator layer 206. In some examples, the various dielectric layers of FIG. 5B and the passivation layer 599 may extend along a length of the radiator layer 206, but the various dielectric layers of FIG. 5B and the passivation layer 599 also may include multiple, non-continuous segments. In some examples, the electrodes 555 and 556 do not extend along the full length of the radiator layer 206, and rather have abbreviated lengths relative to the radiator layer 206, as shown. In some examples, the metal members 558 and 560 do not extend along the full length of the radiator layer 206, and rather have abbreviated lengths relative to the radiator layer 206, as shown.

A wave control structure 562 is a portion of the radiator layer 206 that includes a portion of the piezoelectric layer 554, the pair of electrodes 555 and 556, the pair of metal members 558 and 560, and dielectric layers achieving separation of the electrodes 555, 556, the metal members 558, 560, and the piezoelectric layer 554 from each other in the manner shown. The wave control structure 562 has an arched shape because the piezoelectric layer 554, the pair of metal members 558, 560, the passivation layer 599, and various dielectric layers in the radiator layer 206 have an arched shape. For example, the metal member 560 includes a portion 563 located directly above a corresponding portion 564 of the surface 550; the metal member 558 includes a portion 565 located directly above the portion 564 of the surface 550 and directly below the portion 563 of the metal member 560; the metal member 560 includes a portion 566 located directly above a corresponding portion 567 of the surface 550; the metal member 558 includes a portion 568 located directly above the portion 567 and directly below the portion 566; the metal member 560 includes a portion 569 located directly above a corresponding portion 570 of the surface 550; and the metal member 558 includes a portion 571 directly above the portion 570 and directly below the portion 569. In examples, a distance between the portions 566 and 567 is greater than a distance between the portions 563 and 564. In examples, a distance between the portions 566 and 567 is greater than a distance between the portions 569 and 570. In examples, the distance between the portions 563 and 564 is different than the distance between the portions 569 and 570 (e.g., within 10% of each other), and in some examples, the distance between the portions 563 and 564 and the distance between the portions 569 and 570 are the same. In examples, a distance between the portions 568 and 567 is greater than a distance between the portions 565 and 564. In examples, a distance between the portions 568 and 567 is greater than a distance between the portions 571 and 570. In examples, the distance between the portions 565 and 564 is different than the distance between the portions 571 and 570, and in some examples, the distance between the portions 565 and 564 and the distance between the portions 571 and 570 are the same. In examples, the piezoelectric layer 554 and the passivation layer 599 are arched-shaped similarly to the metal members 558 and 560.

The radiator layer 206 includes a wave control structure 572. The wave control structure 572 includes the piezoelectric layer 554, the passivation layer 599, and the various dielectric layers 551-553, 557, 559, and 561. The wave control structure 572 may also include a pair of electrodes 573 and 574 on opposing sides of the piezoelectric layer 554, as shown. The wave control structure 572 may also include a pair of metal members 575 and 576 above the electrode 574, as shown. As shown, the piezoelectric layer 554, the electrodes 573 and 574, the metal members 575 and 576, the passivation layer 599, and the dielectric layers 557, 559, and 561 have arched shapes as described above. In examples, the lengths of the electrodes 573 and 574 (e.g., ranging from 2 microns to 50 microns in length) are lesser than the lengths of the electrodes 555 and 556 (e.g., ranging from 40 microns to 90 microns in length), respectively. In examples, the lengths of the metal members 575 and 576 (e.g., ranging from 2 microns to 50 microns in length) are lesser than the lengths of the metal members 558 and 560 (e.g., ranging from 2 microns to 50 microns in length), respectively.

In operation, the controller 104 (FIG. 1) excites the pairs of electrodes 555, 556 and the pairs of electrodes 573, 574, which, in turn, excite the piezoelectric layer 554. The piezoelectric layer 554 provides acoustic waves responsive to excitation by the pairs of electrodes 555, 556 and 573, 574. The acoustic waves propagate along the length of the radiator layer 206. The controller 104 may excite the electrodes 555, 556 and 573, 574 in any suitable manner to generate the acoustic waves. In examples, the electrodes 555 and 573 are coupled to ground, and the controller 104 repeatedly excites the electrodes 556 and 574 to generate the acoustic waves. In examples, the electrodes 556 and 574 are coupled to ground and the controller 104 repeatedly excites the electrodes 555 and 573 to generate the acoustic waves, although the remainder of this discussion assumes the electrodes 555 and 573 are coupled to ground and the controller 104 excites the electrodes 556 and 574. The amplitude of the acoustic waves depends on the voltages the controller 104 applies to the pairs of electrodes 555, 556 and 573, 574. The frequency and wavelength of the acoustic waves depends on the frequency with which the controller 104 excites the pairs of electrodes 555, 556 and 573, 574. Although this description assumes excitation of the pairs of electrodes 555, 556 and 573, 574 that are specifically shown in FIG. 5B, in examples, the controller 104 excites some or all of the pairs of electrodes in the sets of wave control structures 500, 502, and 504 (FIG. 5A) in any suitable pattern (e.g., in an alternating pattern, in a serial pattern, in groups), with any suitable frequency and any suitable voltage, to produce acoustic waves having target characteristics such as amplitude and frequency.

Still referring to FIG. 5B, the radiator layer 206 resonates at an operating frequency by reflecting acoustic waves having the operating frequency and attenuating acoustic waves having other frequencies. In examples, the operating frequency ranges from 100 MHz to 300 MHz, inclusive. In examples, the operating frequency ranges from 200 MHz to 300 MHz, inclusive. The arched structures of the radiator layer 206 (e.g., the arched piezoelectric layer 554, the arched metal members 558, 560, 575 and 576, and the arched dielectrics 557, 559 and 561 in the wave control structures 562, 572) facilitate reflection of acoustic waves within the operating frequency range because they are sized to be larger than the wavelengths of the acoustic waves, which causes greater contrast in the radiator layer 206 geometric characteristics (e.g., contrast in size between wave control structures of the radiator layer 206 and portions of the radiator layer 206 that do not include wave control structures) and creates larger reflections. The arched shape of the structures in the radiator layer 206 provides large perturbations in the pathway along which the acoustic waves propagate, thereby enabling strong acoustic wave reflections. These arched structures of the radiator layer 206 also facilitate reflection of acoustic waves over a relatively large bandwidth (e.g., 100 MHz or more) because they are geometrically large structures (relative the acoustic wave wavelengths) and thus are able to reflect a greater number of acoustic wave wavelengths, covering a relatively large frequency band.

FIG. 5C is a top-down view of electrically active structures in the radiator layer 206, in accordance with various examples. Specifically, FIG. 5C shows multiple bond pads 202, which are also shown in FIGS. 2-5A. As described, the bond pads 202 are located on a device side of the semiconductor die 106 so as to be accessible to bond wires, solder balls, etc. that couple the bond pads 202 to other structures, such as structures on the PCB 102. FIG. 5C also shows multiple electrodes in the radiator layer 206. Specifically, FIG. 5C shows a ground electrode 580 that extends along a length of the radiator layer 206, and FIG. 5C also shows a six sets of electrodes 582, 584, 586, 588, 590, and 592. Electrodes 582 are electrically coupled together; electrodes 582 are electrically coupled together; electrodes 586 are electrically coupled together; electrodes 588 are electrically coupled together; electrodes 590 are electrically coupled together; and electrodes 592 are electrically coupled together. The electrodes of one set of electrodes may be arranged in an interleaving pattern with electrodes of another set of electrodes. For example, the sets of electrodes 582 and 588 have interleaving electrodes; the sets of electrodes 584 and 590 have interleaving electrodes; and the sets of electrodes 586 and 592 having interleaving electrodes. Such an interleaving pattern facilitates excitation in specific patterns, such as on an alternating basis, although the controller 104 may excite the various electrodes in any suitable pattern, as described above. Each of the sets of electrodes 582, 584, 586, 588, 590, and 592 is coupled to a different bond pad 202 through which the controller 104 (FIG. 1) may individually excite a respective set of electrodes. Thus, the controller 104 may excite the electrodes in the set of electrodes 586 in an alternating pattern with the electrodes in the set of electrodes 592. Further, the ground electrode 580 may be coupled to one or more bond pads 202. Although FIG. 5C shows the ground electrode 580 as being a continuous piece of conductive material extending along a length of the radiator layer 206, the ground electrode may be divided into multiple discontinuous segments, such as the electrodes 555 and 573 shown in FIG. 5B. Any and all such configurations for the electrodes are possible and included in the scope of this description.

The bond pads 202, the ground electrode 580, and the sets of electrodes 582, 584, 586, 588, 590, and 592 may be located on different vertical levels of the semiconductor die 106. For example, the bond pads 202 may be located on the device side of the semiconductor die 106, while the ground electrode 580 may be located in the radiator layer 206, whether above, below, or on a same vertical level as the bond pads 202. Similarly, the sets of electrodes 582, 584, 586, 588, 590, and 592 may be located in the radiator layer 206, whether above, below, or on a same vertical level as the bond pads 202. In examples, and as shown in FIG. 5B, the ground electrode 580 may be located on a different vertical level than the sets of electrodes 582, 584, 586, 588, 590, and 592, with the piezoelectric layer 554 positioned between (i) the ground electrode 580 and (ii) the sets of electrodes 582, 584, 586, 588, 590, and 592. Electrically active structures, such as electrodes and bond pads, that are on different vertical levels may be coupled to each other as appropriate with conductive (e.g., copper) vias.

FIG. 5D is a top-down view of the piezoelectric layer 554 in the radiator layer 206, in accordance with various examples. In examples, the piezoelectric layer 554 may have a length that extends along a length of the radiator layer 206 and a width that is the same as that of the electrodes in the sets of electrodes 582, 584, 586, 588, 590, and 592 (FIG. 5C), although the dimensions of the piezoelectric layer 554 may vary.

FIG. 5E is a top-down view of metal members 594 in the radiator layer 206, in accordance with various examples. The metal members 594 are representative of the upper metal members shown in FIG. 5B, such as the metal members 560 and 576. The metal members 594 corresponding to the wave control structures 500 (FIG. 5A) may have a smaller pitch and the metal members 594 corresponding to the wave control structures 502 and 504 (FIG. 5A) may have a larger pitch.

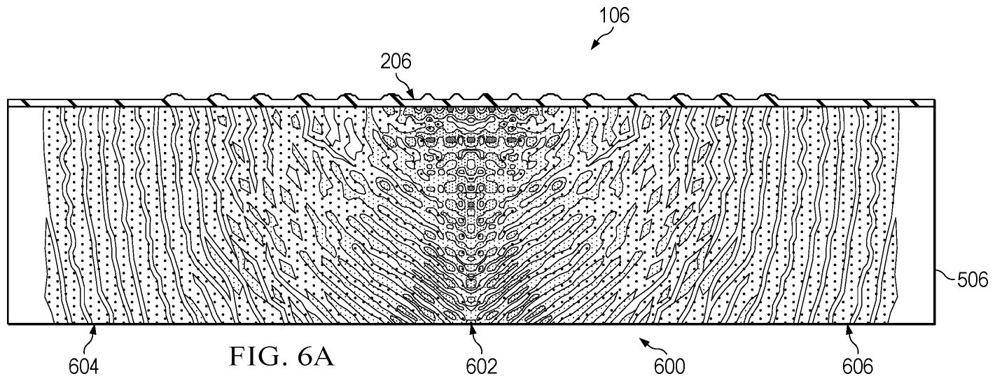

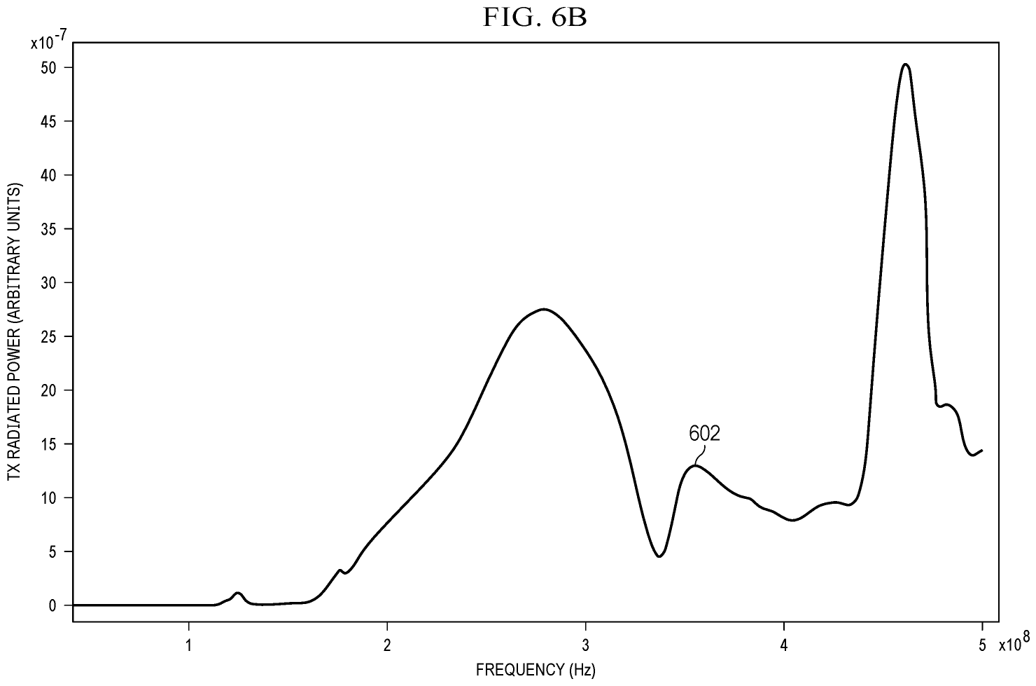

FIG. 6A is a cross-sectional schematic diagram of the semiconductor die 106 and an acoustic wave emission pattern of the semiconductor die 106, in accordance with various examples. As shown, the radiator layer 206 provides acoustic waves that propagate through the semiconductor substrate 506 and toward an external area 600. The acoustic waves are strongest in a central portion 602 of the semiconductor substrate 506 and are weaker in lateral areas 604 and 606 of the semiconductor substrate 506.

FIG. 6B is a graph showing radiated power of acoustic waves from the radiator layer 206 as a function of operating frequency, in accordance with various examples. The x-axis shows acoustic wave frequency in Hertz and the y-axis shows acoustic wave radiated power in any suitable unit, such as Watts. Although the radiated power is expressed in arbitrary units, the magnitude of the radiated power over the approximate range of 200 MHz to 300 MHz and over a wide band of approximately 100 MHz is notable. The radiator layer 206 achieves relatively strong output power for acoustic waves in the relatively low frequency range of 200 MHz to 300 MHz, inclusive, although the graph is merely illustrative and output power may be increased in other frequency ranges depending on the specific structural geometries implemented in the radiator layer 206. The structure of the radiator layer 206 is configured to provide Increased output power in the relatively low frequency range of 200 MHz to 300 MHz, inclusive, as described above.

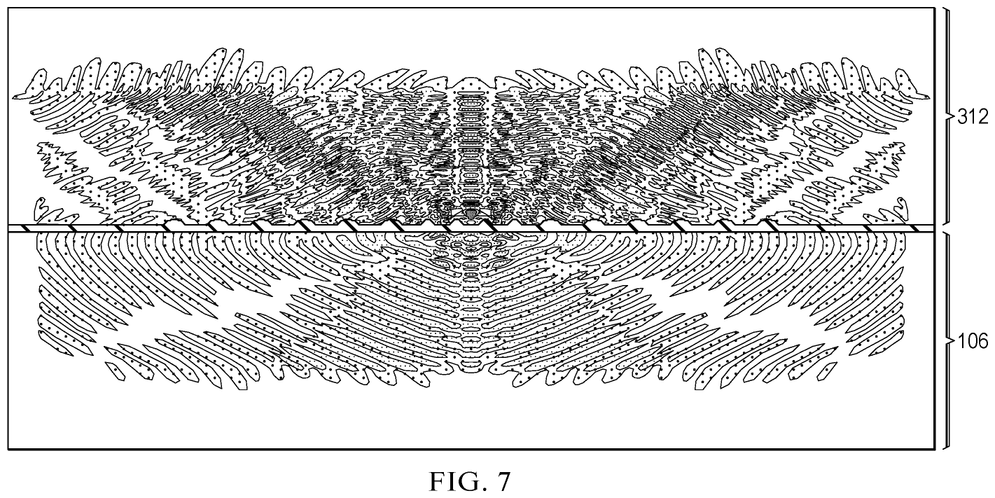

The presence of the mold compound 312 (FIG. 3) may cause the acoustic wave emission pattern to vary from that shown in FIG. 6A. FIG. 7 is a cross-sectional schematic diagram of the mold compound 312 on the semiconductor die 106 and an acoustic wave emission pattern through the semiconductor die 106 and the mold compound 312, in accordance with various examples. As shown, the emission pattern is extended laterally in the mold compound 312 relative to the emission pattern in the semiconductor die 106.

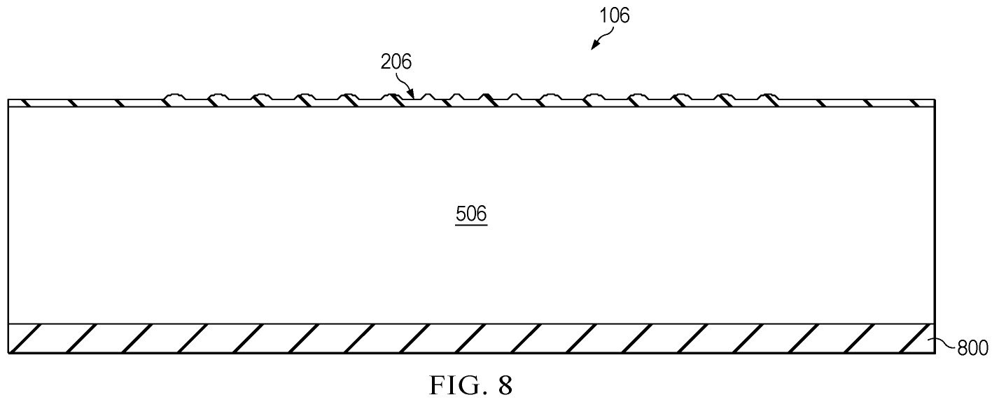

FIG. 8 is a cross-sectional schematic diagram of the semiconductor die 106 on a matching layer 800 and having a radiator layer 206, in accordance with various examples. The matching layer 800 is configured to perform impedance matching between the semiconductor substrate 506 and a medium on an opposite side of the matching layer 800 from the semiconductor substrate 506, such as human tissue or fluid. The impedance gradient of the matching layer 800 may be adjusted as appropriate to achieve a target range of acoustic wave reflections and insertion losses. For example, the impedance gradient of the matching layer 800 may be controlled to reduce insertion losses.

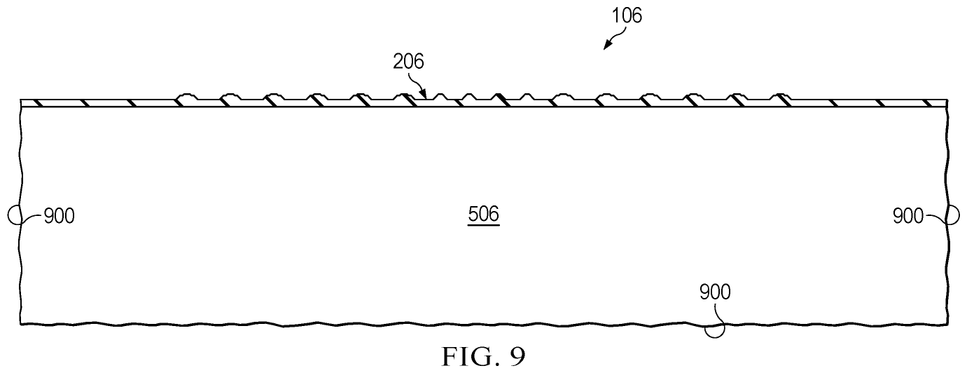

FIG. 9 is a cross-sectional schematic diagram of the semiconductor die 106 having roughened surfaces 900, in accordance with various examples. The roughened surfaces 900 mitigate acoustic wave reflections due to sharp impedance gradients (e.g., between the semiconductor substrate 506 and a medium abutting the semiconductor substrate 506), thereby mitigating insertion losses. In examples, the roughness of the roughened surfaces 900 (e.g., height of ridges on the roughened surfaces 900) has a range that is comparable to the acoustic wave wavelength in the semiconductor substrate 506 (e.g., ranging from 90% of the wavelength to 500% of the wavelength), as this would provide adequate acoustic wave dispersion to scatter the acoustic waves. The roughness of the roughened surface 900 becomes larger at smaller frequencies.

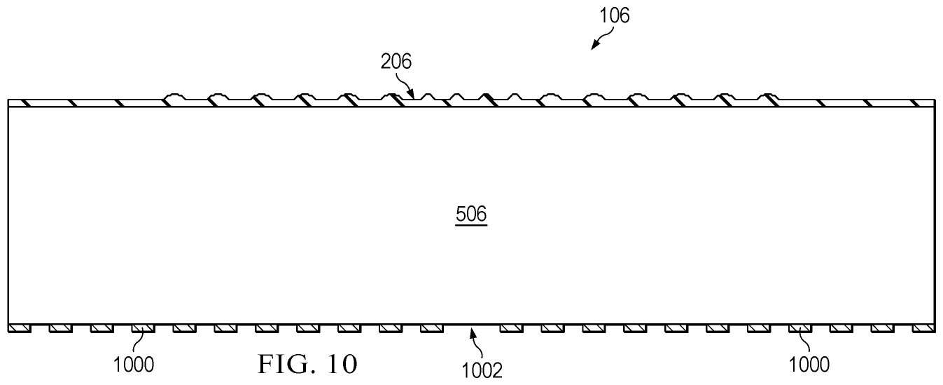

FIG. 10 is a cross-sectional schematic diagram of the semiconductor die 106 having a patterned metal layer 1000 on a non-device side of the semiconductor die 106, in accordance with various examples. The patterned metal layer 1000 is configured to manipulate the acoustic waves propagating through the semiconductor substrate 506. For example, the patterned metal layer 1000 may be patterned to include an opening 1002 to collimate the acoustic waves exiting the semiconductor substrate 506. In examples, the patterned metal layer 1000 may be patterned to mitigate acoustic wave reflections and attendant insertion losses.

In this description, the term “couple” may cover connections, communications, or signal paths that enable a functional relationship consistent with this description. For example, if device A generates a signal to control device B to perform an action: (a) in a first example, device A is coupled to device B by direct connection; or (b) in a second example, device A is coupled to device B through intervening component C if intervening component C does not alter the functional relationship between device A and device B, such that device B is controlled by device A via the control signal generated by device A.

A device that is “configured to” perform a task or function may be configured (e.g., programmed and/or hardwired) at a time of manufacturing by a manufacturer to perform the function and/or may be configurable (or reconfigurable) by a user after manufacturing to perform the function and/or other additional or alternative functions. The configuring may be through firmware and/or software programming of the device, through a construction and/or layout of hardware components and interconnections of the device, or a combination thereof.

Uses of the term “ground” or variants thereof in the foregoing description may include a chassis ground, an Earth ground, a floating ground, a virtual ground, a digital ground, a common ground, and/or any other form of ground connection applicable to, or suitable for, the teachings of this description. Unless otherwise stated, “about,” “approximately,” or “substantially” preceding a parameter means being within +/−10 percent of that parameter. Modifications are possible in the described examples, and other examples are possible within the scope of the claims.

What is claimed is:

1. A semiconductor die, comprising:
a semiconductor substrate having a surface, the surface having first and second surface portions; and
a radiator layer on the surface, the radiator layer comprising:
a metal member having a first metal member portion above the first surface portion and a second metal member portion above the second surface portion, a first distance between the first metal member portion and the first surface portion, and a second distance between the second metal member portion and the second surface portion, the first distance less than the second distance;
first and second electrodes; and
a piezoelectric layer extending along a length of the radiator layer and on each of the first and second electrodes, the piezoelectric layer between the first and second metal members and the semiconductor substrate.

2. The semiconductor die of claim 1, wherein the radiator layer is configured to emit an acoustic wave having a frequency in the range of 100 MHz to 300 MHz, inclusive.

3. The semiconductor die of claim 2, wherein the acoustic wave is at least 100 MHz wide.

4. The semiconductor die of claim 2, wherein the radiator layer is configured to expand and contract responsive to the acoustic wave.

5. The semiconductor die of claim 1, wherein the metal member includes a third metal member portion, the second metal member portion is between the first and third metal member portions, the surface has a third surface portion, the second surface portion is between the first and third surface portions, and a third distance between the third metal member portion and the third surface portion is less than the second distance.

6. The semiconductor die of claim 1, wherein the first and second electrodes have a first pitch therebetween which differs from a second pitch between the second electrode, and a third electrode, and the first, second and third electrodes are on a common surface of the piezoelectric layer.

7. An electronic device, comprising:
a printed circuit board (PCB); and
a semiconductor die coupled to the PCB, the semiconductor die comprising:
a semiconductor substrate including a surface having a first surface portion, a second surface portion, and a middle surface portion between the first and second surface portions; and
a radiator layer on the surface, the radiator layer having an acoustic wave control structure, the acoustic wave control structure comprising:
a metal member having a first metal member portion, a second metal member portion and a middle metal member portion between the first and second metal member portions, the first, middle, and second metal member portions above the first, middle, and second surface portions, respectively, each of a first distance between the first metal member portion and the first surface portion and a second distance between the second metal member portion and the second surface portion less than a third distance between the middle metal member portion and the middle surface portion;
a piezoelectric layer between the metal member and the semiconductor substrate;
first and second electrodes on opposing surfaces of the piezoelectric layer and coupled to the PCB; and
a dielectric layer on the metal member.

8. The electronic device of claim 7, wherein the first distance is within 10% of the second distance.

9. The electronic device of claim 7, wherein a length of the metal member is in a range from 2 microns to 90 microns, inclusive.

10. The electronic device of claim 7, wherein the first and second electrodes are configured to excite the piezoelectric layer to produce an acoustic wave, and the acoustic wave has a frequency in a range from 100 MHz to 300 MHz, inclusive.

11. The electronic device of claim 10, wherein the acoustic wave is a first acoustic wave, and the metal member is configured to reflect the first acoustic wave and to attenuate second acoustic waves having frequencies outside of the 100 MHz to 300 MHz range.

12. The electronic device of claim 10, wherein the radiator layer is configured to expand and contract responsive to the acoustic wave.

13. The electronic device of claim 7, further comprising third and fourth electrodes on a same surface of the piezoelectric layer as the first electrode, wherein a first pitch between the first and third electrodes differs from a second pitch between the third and fifth electrodes.

14. The electronic device of claim 7, wherein the PCB includes an orifice, and the semiconductor die is configured to emit and receive acoustic waves via the orifice.

15. The electronic device of claim 7, further comprising a mold compound on the semiconductor die, wherein the radiator layer is configured to emit acoustic waves via the mold compound.

16. A semiconductor die, comprising:
a semiconductor substrate; and
a radiator layer on the semiconductor substrate, the radiator layer comprising:
first and second metal members;
first and second electrodes; and
a piezoelectric layer having opposite first and second sides, in which the piezoelectric layer is between the first and second electrodes, and the first side faces the semiconductor substrate, and the second side faces the first and second metal members, and the first and second electrodes are configured to produce an acoustic wave by exciting the piezoelectric layer, and the first and second metal members are configured to reflect the acoustic wave responsive to the acoustic wave having a frequency within a target frequency band.

17. The semiconductor die of claim 16, wherein the target frequency band includes 100 MHz to 300 MHz, inclusive.

18. The semiconductor die of claim 16, wherein the target frequency band is at least 100 MHz wide.
